(19) United States
(12) Patent Application Publication
(10) Pub. No.: US 2026/0279519 A1
(43) Pub. Date: Sep. 17, 2026

(54) CLINICAL TRIAL PROPOSAL DEVICE AND CLINICAL TRIAL PROPOSAL METHOD

(71) Applicant: HITACHI HIGH-TECH CORPORATION, Tokyo (JP)

(72) Inventors: Etsuko KAMIKUMASU, Tokyo (JP); Koichiro KIMOTSUKI, Tokyo (JP); Aya KURIKI, Tokyo (JP)

(73) Assignee: HITACHI HIGH-TECH CORPORATION, Tokyo (JP)

(21) Appl. No.: 19/567,656

(22) Filed: Mar. 16, 2026

(30) Foreign Application Priority Data
Mar. 17, 2025 (JP) 2025-042240

(51) Int. Cl. G16H 10/20; G16H 10/60

(52) U.S. Cl. CPC G16H 10/20; G16H 10/60

(57) ABSTRACT

A clinical trial proposal device according to the invention includes: a reception unit configured to receive a clinical trial condition including a plurality of items required of a subject of a clinical trial, and patient information including the plurality of items about a plurality of patients who may be the subject; an extraction unit configured to extract, if a value for some item included in the patient information does not exist, a candidate of the subject from the patient information by comparing values for other items existing in the patient information with values for other items in the clinical trial condition, and display the extracted candidate of the subject on a screen; and a proposal unit configured to propose the clinical trial for the extracted candidate.

### BACKGROUND OF THE INVENTION

### 1. Field of the Invention

The present invention relates to a clinical trial proposal device and a clinical trial proposal method.

### 2. Description of Related Art

In the field of drug discovery, a new chemical substance must be tested on humans before being approved as a new drug by a government-controlled review and approval organization. Such a test is commonly referred to as a “clinical trial”. In the clinical trial, a patient voluntarily applies to a medical institution or the like to be a subject. A subject who matches a condition for the clinical trial will take the unapproved new drug throughout a period of attendance or admission to a hospital, or at home, based on an instruction from a pharmaceutical company or the like. The medical institution or the like will diagnose the subject before, during, and after the period of taking the drug, and report therapeutic effects and presence or absence of adverse reactions (side effects) to the pharmaceutical company or the like. Recently, it is common for a computer to extract a subject who matches the condition for the clinical trial from among a large number of patients.

A subject candidate extraction system in PTL 1 extracts a subject who conforms to a clinical trial protocol from electronic medical record information of a plurality of patients, using reference information in the clinical trial protocol. A hospital information system in PTL 2 selects an investigational drug and/or unapproved drug suitable for a patient based on personal information and medical information about the patient, and information on the investigational drug and the unapproved drug. A medical system in PTL 3 selects a patient to be guided to a clinical trial facility based on selection criteria for selecting a patient to be guided to the clinical trial facility where a clinical trial is conducted and patient information that specifies information on the patient.

### CITATION LIST

### Patent Literature

PTL 1: JP2024-28423A
PTL 2: JP2019-75046A
PTL 3: JP2018-206287A

### SUMMARY OF THE INVENTION

It is important to extract a subject who truly conforms to a condition for a clinical trial from as many populations as possible. For example, it is expected that the number of opportunities for patients in cancer genome medical treatment to undergo cancer gene panel testing will increase in the future. There is a need to extract an eligible patient to be a subject from among these patients based on a result of cancer gene panel testing.

However, none of the subject candidate extraction system of PTL 1, the hospital information system in PTL 2, and the medical system in PTL 3 consider a case where values for some of items required of patients as the condition for the clinical trial may be missing. As a result, the extract result is inevitably biased towards the safe side, and subjects will be left out of the extraction when there is no practical problem even though values for certain items are missing, or when the values for the items can be supplemented later. As a result, a cancer patient will lose out on treatment opportunities including clinical trials. Therefore, an object of the invention is to accurately extract a subject even when a part of patient information is missing.

A clinical trial proposal device according to the invention is characterized by including: a reception unit configured to receive a clinical trial condition including a plurality of items required of a subject of a clinical trial, and patient information including the plurality of items about a plurality of patients who may be the subject; an extraction unit configured to extract, if a value for some item included in the patient information does not exist, a candidate of the subject from the patient information by comparing values for other items existing in the patient information with values for other items in the clinical trial condition, and display the extracted candidate of the subject on a screen; and a proposal unit configured to propose the clinical trial for the extracted candidate.

Other means will be described in an embodiment for carrying out the invention.

The invention can accurately extract a subject even when a part of patient information is missing.

### BRIEF DESCRIPTION OF THE DRAWINGS

FIG. 1 is a diagram illustrating a configuration and the like of a clinical trial proposal device;

FIG. 2A is an example of clinical trial information;

FIG. 2B is an example of a clinical trial condition;

FIG. 2C is an example of patient information;

FIG. 2D is an example of weight information;

FIG. 2E is an example of conformance score information;

FIG. 2F is an example of clinical trial proposal information;

FIG. 3 is a flowchart of an overall processing procedure;

FIG. 4 is a flowchart of a registration processing procedure;

FIG. 5 is a flowchart of a patient retrieval processing procedure;

FIG. 6 is a flowchart of an additional input processing procedure;

FIG. 7 is a flowchart of a clinical trial retrieval processing procedure;

FIG. 8 is a flowchart of a conformance score recalculation processing procedure;

FIG. 9 is a flowchart of a PS estimation processing procedure;

FIG. 10 is an example of a clinical trial registration screen;

FIG. 11 is an example of a patient retrieval result screen;

FIG. 12 is an example of a clinical trial proposal screen; and

FIG. 13 is an example of a clinical trial retrieval result screen.

### DESCRIPTION OF EMBODIMENTS

Hereinafter, an embodiment of the invention (referred to as “the present embodiment”) will be described in detail with reference to the drawings. The present embodiment is an example in which a subject of a clinical trial is extracted from patients who have undergone gene panel testing for cancer genome medical treatment. However, the invention is also applicable to medical fields other than cancer genome medical treatment.

### Terms and the Like

A clinical trial refers to a clinical test conducted on humans with an aim of obtaining approval for a new drug. The clinical trial includes three development phases, “Phase 1 trial”, “Phase 2 trial”, and “Phase 3 trial”. Phase 1 trial is conducted mainly for adverse reactions of a new drug. Phase 2 trial is conducted mainly for adverse reactions and effects of a new drug. Phase 3 trial is conducted on a larger number of patients and for comparing effects of a new drug with effects of a standard treatment (existing approved treatment) and with effects of a placebo.

A subject refers to a patient who is a subject of a clinical trial. The patient here includes a healthy person who is under care of a medical institution, even though he or she is not currently suffering from any disease. The patient is informed by a medical institution of benefits (effects, compensation, and the like) and disadvantages (adverse reactions and the like) of the clinical trial, and then voluntarily agree to be a subject. A subject may withdraw from being a subject at any time for any reason. Phase 1 trial is often conducted mainly on healthy people, while Phase 2 trial and 3 trial are often conducted mainly on unhealthy patients.

A pharmaceutical company refers to a company that produces new drugs and an entity that submits applications to the government.

A medical institution refers to a person (either an individual or a juridical person) who provides treatment to the subject.

Cancer genome medical treatment refers to a medical treatment conducted on a cancer patient, which is individualized based on genetic information of cells of a diseased part.

Gene panel testing refers to a test that detects genetic changes in cells in the diseased part as a part of the cancer genome medical treatment. Genetic changes appear as changes in the DNA base sequence within chromosomes.

A clinical trial condition refers to a condition required of a subject by a clinical trial of a new drug. The clinical trial condition includes a plurality of items such as carcinoma, treatment period, gender, age, whether metastasis has occurred, whether chemotherapy has been administered, driver genes, and performance status (PS). A specific value is set for each item. For example, a value of “stomach cancer” or the like is set for the item of “carcinoma”. A value of “yes” or “no” is set for the item of “whether metastasis has occurred”. The clinical trial condition is designated by the pharmaceutical company.

Patient information refers to medical information about a patient. The patient information does not include information that can identify an individual, such as a name. The patient information includes a plurality of items. These items generally correspond to the items included in the clinical trial condition. A specific value is set and diagnosed for each item. The patient information is acquired by the medical institution that performs examination and the like on the patient.

### Conformance Score

It is now assumed that the clinical trial condition for all new drugs includes m items, and the patient information for all patients also includes m items arranged in the same order as the clinical trial condition. If the value for the nth (n=1, 2, 3, . . . , m) item in the clinical trial condition matches the value for the nth item in the patient information, a matching degree Pn=1, otherwise, the matching degree Pn=0. In this case, for a combination of a clinical trial x and a patient y, a conformance score Sxy is defined as a weighted sum of the matching degrees as follows.

Sxy=ΣWn·Pn

Here, Wn is a weight, and as a simple example, “ΣWn=1” and “0≤Wn≤1”. In other words, the conformance score is a numerical representation of a degree to which the clinical trial condition for a new drug conforms to the patient information of a certain patient. A patient with a high conformance score is suitable as a subject for the new drug.

If a value for the jth item in the patient information does not exist, a matching degree Pj=0. In this case, Wj·Pj=0, but even in this case, ΣWn·Pn is not necessarily 0. In this way, even when values for some of the items included in the patient information do not exist, it is possible to extract a candidate of the subject from the patient information by comparing values for other items existing in the patient information with values for other items in the clinical trial condition. Then, if the weights are corrected, the conformance score can be recalculated based on the weights after correction.

### Configuration and the Like of Clinical Trial Proposal Device

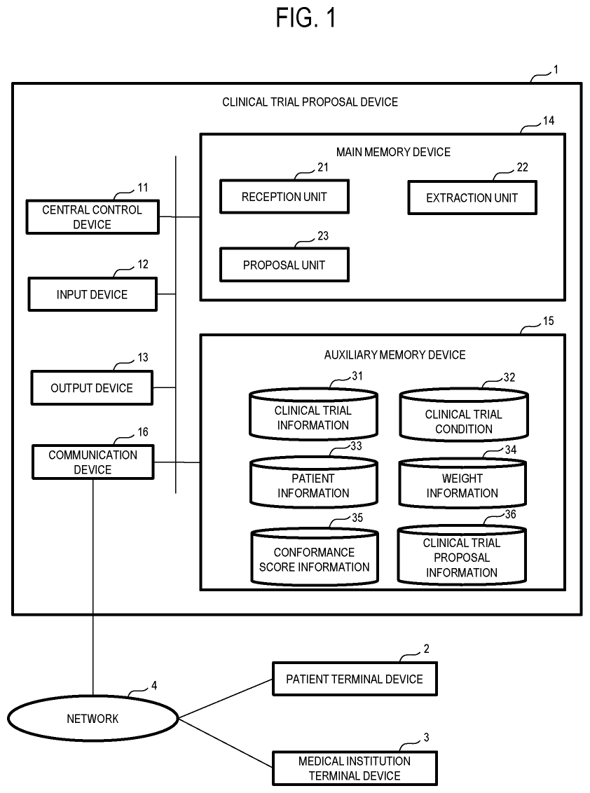

FIG. 1 is a diagram illustrating a configuration and the like of a clinical trial proposal device 1. The clinical trial proposal device 1 is a general computer, and includes a central control device 11, an input device 12 such as a mouse or a keyboard, an output device 13 such as a display, a main memory device 14, an auxiliary memory device 15, and a communication device 16. These elements are connected to each other via a bus. The auxiliary memory device 15 stores clinical trial information 31, clinical trial condition 32, patient information 33, weight information 34, conformance score information 35, and clinical trial proposal information 36 (which will be described in detail below).

A reception unit 21, an extraction unit 22, and a proposal unit 23 in the main memory device 14 are programs. In the following description, when a processing entity is described as “XX unit”, it means that the central control device 11 implements the function of each program (which will be described in detail below) by reading these programs from the auxiliary memory device 15 to the main memory device 14.

The clinical trial proposal device 1 is connected to a patient terminal device 2 and a medical institution terminal device 3 via a wired or wireless network 4. The patient terminal device 2 and the medical institution terminal device 3 are portable or stationary computers. Some or all of the information 31 to 36 stored in the auxiliary memory device 15 and some or all of the programs 21 to 23 read into the main memory device 14 may be stored in a distributed manner across a plurality of housings. The pharmaceutical company operates the clinical trial proposal device 1 or a pharmaceutical company terminal device (not illustrated). The candidate (patient) of the subject operates the patient terminal device 2. The medical institution operates the medical institution terminal device 3.

### Clinical Trial Information

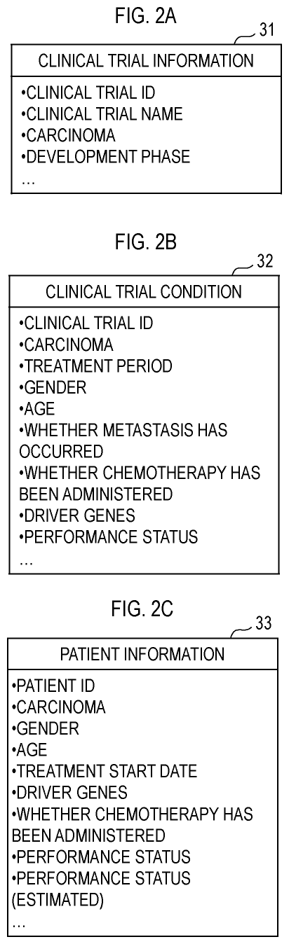

FIG. 2A is an example of the clinical trial information 31. The clinical trial information 31 stores a clinical trial name, carcinoma, development phase, and the like in association with a clinical trial ID (identifier). There are as many pieces of clinical trial information 31 as there are clinical trials.

### Clinical Trial Condition

FIG. 2B is an example of the clinical trial condition 32. The clinical trial condition 32 stores a plurality of items and values therefor in association with the clinical trial ID. Specifically, as mentioned above, the items refer to carcinoma, treatment period, gender, age, whether metastasis has occurred, whether chemotherapy has been administered, driver genes, PS, and the like. There are as many clinical trial conditions 32 as there are clinical trials. Driver genes refer to genes that cause the cancer and states thereof. PS refers to an overall health degree of the patient and is a different concept from the progress (stage) of cancer.

### Patient Information

FIG. 2C is an example of the patient information 33. The patient information 33 stores a plurality of items and values therefor in association with a patient ID (the name or the like cannot be specified). The items basically correspond to the items in the clinical trial condition 32. However, these items do not necessarily completely correspond to the items in the clinical trial condition 32. For example, the item “whether metastasis has occurred” that exists in the clinical trial condition 32 may not exist in the patient information 33 (which will be described in detail below). Furthermore, “PS” existing in the clinical trial condition 32 is a confirmed value after diagnosis, but in the patient information 33, the confirmed value may not exist, and instead an estimated value may exist. There are as many pieces of patient information 33 as there are patients.

### Weight Information

FIG. 2D is an example of the weight information 34. The weight information 34 stores a weight (Wn) for each item in the clinical trial condition 32 in association with the clinical trial ID. There are as many pieces of weight information 34 as there are clinical trials.

### Conformance Score Information

FIG. 2E is an example of the conformance score information 35. The conformance score information 35 stores the conformance score (Sxy) and the like in association with a combination of the patient ID and the clinical trial ID. There are as many pieces of conformance score information 35 as there are combinations of patients and clinical trials.

### Clinical Trial Proposal Information

FIG. 2F is an example of the clinical trial proposal information 36. The clinical trial proposal information 36 stores whether additional input has been made, status, whether to accept, reason, and the like, in association with the combination of the clinical trial ID and the patient ID. There are as many pieces of clinical trial proposal information 36 as there are combinations of clinical trials and patients.

### Overall Processing Procedure

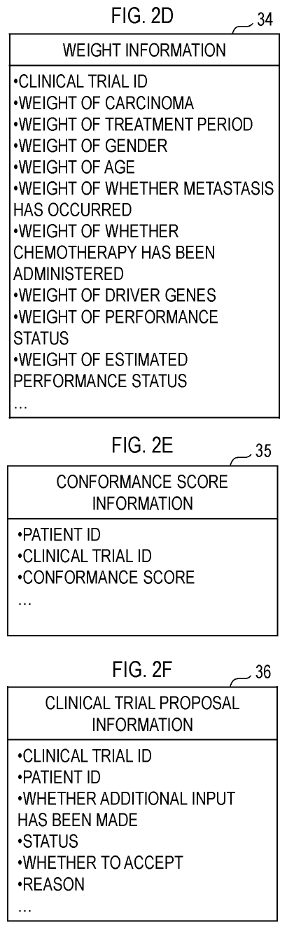

FIG. 3 is a flowchart of an processing procedure. As a premise for starting the overall processing procedure, it is assumed that the patient information 33 for a large number of patients has already been stored in the auxiliary memory device 15 with values assigned to the items. That is, the reception unit 21 receives a large number of pieces of patient information 33 via the medical institution terminal device 3 and stores the received result in the auxiliary memory device 15. However, every piece of patient information 33 does not necessarily store values for all items.

In step S101, the reception unit 21 receives registration of a clinical trial. Thereafter, the flow of processing branches into two paths.

In step S102, the extraction unit 22 or the like retrieves a patient starting from the clinical trial. That is, the extraction unit 22 or the like retrieves a relevant patient using the clinical trial as a retrieval key.

In step S103, the proposal unit 23 proposes inputting additional information.

In step S103b, the proposal unit 23 receives confirmation of the proposed result from the medical institution. The overall processing procedure then ends.

In step S104, the extraction unit 22 or the like retrieves a clinical trial starting from the patient. That is, the extraction unit 22 or the like retrieves a relevant clinical trial using the patient as a retrieval key. The overall processing procedure then ends.

### Registration Processing Procedure

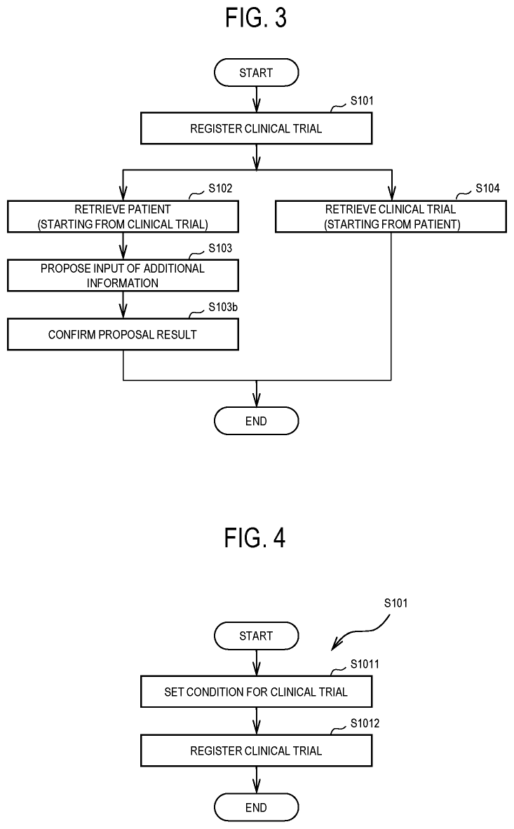

FIG. 4 is a flowchart of a registration processing procedure. The registration processing procedure is a detailed description of step S101 in FIG. 3.

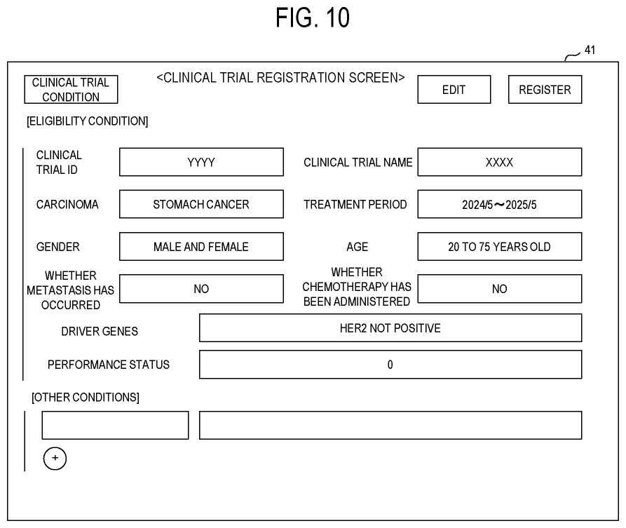

In step S1011, the reception unit 21 receives the setting of the clinical trial condition set by the pharmaceutical company. Specifically, the reception unit 21 displays a clinical trial registration screen 41 (FIG. 10) on the output device 13 and receives information input by the pharmaceutical company into blanks on the clinical trial registration screen 41 via the input device 12. Although description is being made with reference to FIG. 4, the description will be made below with reference to FIG. 10.

FIG. 10 is an example of the clinical trial registration screen 41. The clinical trial registration screen 41 has fields for inputting values for the items in the clinical trial condition 32 (FIG. 2B). The pharmaceutical company (or a medical institution or a company delegated thereby) inputs the values for the items in each field. The description will be made returning to FIG. 4.

In step S1012, the reception unit 21 stores the information received in step S1011 as the clinical trial information 31 and the clinical trial condition 32 in the auxiliary memory device 15. The registration processing procedure then ends. The registration processing procedure is repeated each time a clinical trial for a new drug is attempted.

### Patient Retrieval Processing Procedure

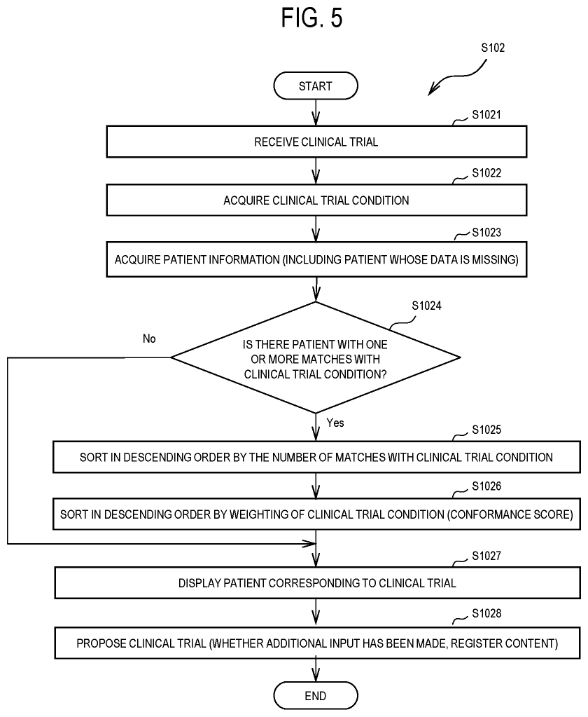

FIG. 5 is a flowchart of a patient retrieval processing procedure. The patient retrieval processing procedure is a detailed description of step S102 in FIG. 3.

In step S1021, the reception unit 21 receives input of a clinical trial as a reference from the pharmaceutical company via the input device 12. When calculating the conformance score of all patients for a single clinical trial as a reference, the clinical trial ID of that clinical trial is referred to as a “key clinical trial ID”. A plurality of key clinical trial IDs may be input. For convenience of description, it is assumed here that 10 key clinical trial IDs are input.

In step S1022, the extraction unit 22 acquires the clinical trial condition 32 corresponding to the key clinical trial ID from the auxiliary memory device 15.

In step S1023, the extraction unit 22 calculates the conformance score for the combination of the key clinical trial ID and the patient ID by comparing each of the 10 clinical trial conditions 32 acquired in step S1022 with the patient information 33 of all patients. The number of calculated conformance scores is equal to 10 times the number of patients registered as the patient information 33. Incidentally, if there is no item in the patient information 33 that matches the value for the item in the clinical trial condition 32, the conformance score will be “0”. The extraction unit 22 stores, for each clinical trial ID, the same number of combinations of the patient ID and the conformance score as the number of patients.

In step S1024, the extraction unit 22 determines whether there is a patient with one or more matches with the clinical trial condition. “One or more matches with the clinical trial condition” means that there is one or more items with matching values (conformance score is not “0”). If there is a patient with one or more matches with the clinical trial condition (step S1024 “YES”), the extraction unit 22 proceeds to step S1025, otherwise (step S1024 “NO”) proceeds to step S1027.

In step S1025, the extraction unit 22 sorts the clinical trial IDs in a descending order of the number of patients with matching values for one or more items in the clinical trial condition. That is, the extraction unit 22 arranges the clinical trial IDs in a descending order of the number of candidates of the subject. For example, if there are 400 patients for the clinical trial ID “001”, 500 patients for the clinical trial ID “002”, and 600 patients for the clinical trial ID “003”, the extraction unit 22 will arrange the clinical trial IDs in the order of “003, 002, 001”. The extraction unit 22 may further sort the 400 patients with the clinical trial ID “001” in order of the number of patient information items that match the clinical trial condition items. For example, the group of patients with matching values for the four items of “carcinoma”, “treatment period”, “gender”, and “age” will be ranked in front of the group of patients with matching values for the three items of “carcinoma”, “treatment period”, and “gender”.

In step S1026, the extraction unit 22 sorts the patient IDs in a descending order of the conformance scores. That is, the extraction unit 22 arranges the patient IDs in a descending order of the conformance scores. In the above example, the extraction unit 22 sorts the 600 patient IDs for the clinical trial ID “003” in a decreasing order of the conformance scores, sorts the 500 patient IDs for the clinical trial ID “002” in a decreasing order of the conformance scores, and sorts the 400 patient IDs for the clinical trial ID “001” in a decreasing order of the conformance scores.

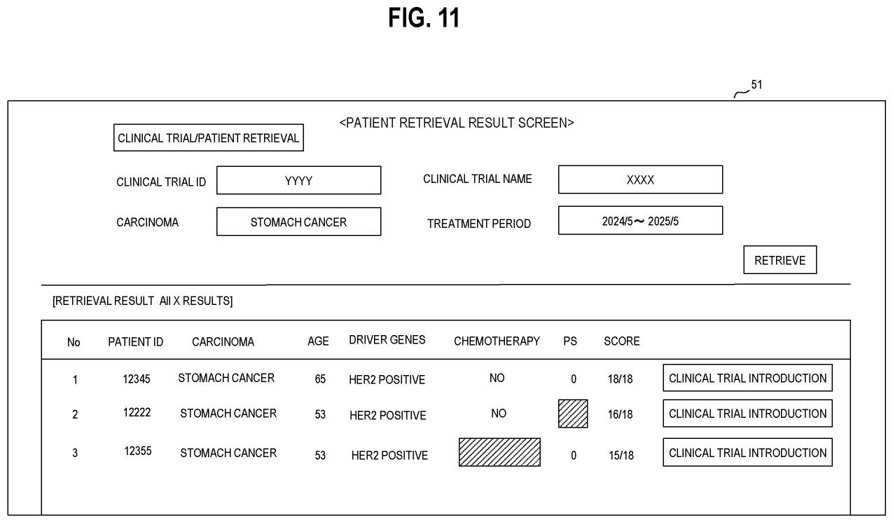

In S1027, the proposal unit 23 displays the patients (candidates of the subject) corresponding to the clinical trial. Specifically, the proposal unit 23 displays a patient retrieval result screen 51 (FIG. 11) on the output device 13. Although description is being made with reference to FIG. 5, the description will be made below with reference to FIG. 11.

FIG. 11 is an example of the patient retrieval result screen 51. The upper part of the patient retrieval result screen 51 displays the key clinical trial ID and information associated therewith. The lower part of the patient retrieval result screen 51 displays a retrieval result. Referring to FIG. 11, for example, the following can be known.
At least three patients have been candidates of the subject for the clinical trial with the clinical trial ID “YYYY”. Note that in actual, an extraordinarily large number of candidates are displayed.
The extraction unit 22 extracts patients with high conformance scores (full marks or close to full marks) as candidates of the subject.
Of the three candidates, the patient “12345” has the highest conformance score (18/18).
The patient “12222” has the second highest conformance score (16/18). The difference thereof in the conformance score with the patient “12345” occurred since the value for the item “PS” did not exist (is missing) in the patient information 33.
The patient “12355” has the third highest conformance score (15/18). The difference thereof in the conformance score with the patient “12222” occurred since the value for the item “whether chemotherapy has been administered” did not exist in the patient information 33 and the weight of that item was relatively large.
Although it cannot be known directly from FIG. 11, the patients displayed at the top may have a greater number of matching items in the clinical trial condition than the patients displayed below.

If the processing goes through the “NO” branch in step S1024, the proposal unit 23 displays “no corresponding results” on the patient retrieval result screen 51. The description will be made returning to FIG. 5.

In step S1028, if there is a missing part in the patient information 33, the proposal unit 23 prompts additional input of the missing part via the medical institution terminal device 3. If there is no missing part in the patient information 33, the proposal unit 23 proposes via the patient terminal device 2 or the medical institution terminal device 3 that the patient being a subject of the clinical trial (proposes the clinical trial for the extracted candidate). Here, “propose” means, for example, displaying a message about that on the screen or sending an email or the like. The patient retrieval processing procedure then ends.

Note that in each step of the patient retrieval processing procedure, the reception unit 21 receives weights of the items via the input device 12, stores the weights as the weight information 34, receives weights after correction, and updates the weight information 34. The extraction unit 22 accesses the weight information 34 to acquire the necessary weights, and stores the calculated conformance scores as the conformance score information 35. If the weights are corrected, the extraction unit 22 recalculates the conformance scores based on the weights after correction and updates the conformance score information 35. The proposal unit 23 stores the content of the proposal, such as the screen output, as the clinical trial proposal information 36. The same applies to the clinical trial retrieval processing procedure described below.

### Additional Input Processing Procedure

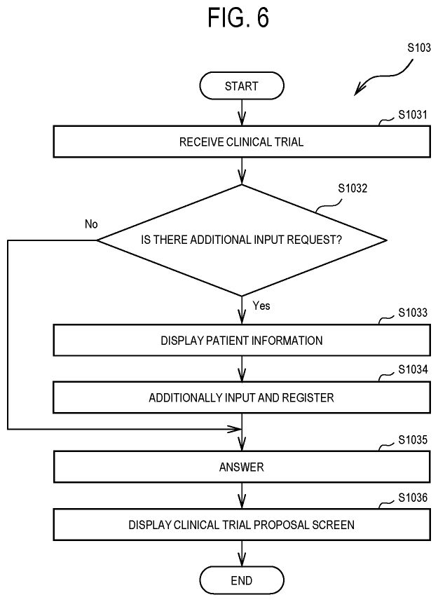

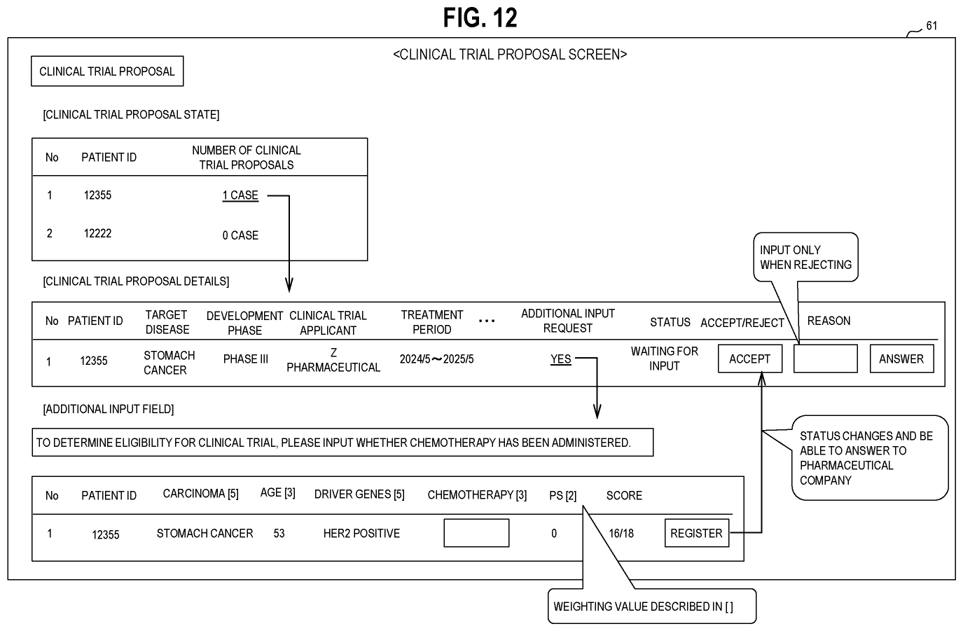

FIG. 6 is a flowchart of an additional input processing procedure. The additional input processing procedure is a detailed description of step S103 in FIG. 3. For ease of understanding, the description will be made with reference to FIG. 12 before FIG. 6.

FIG. 12 is an example of a clinical trial proposal screen 61. Now, the medical institution is looking at the clinical trial proposal screen 61. In the field of “clinical trial proposal state” in the upper part of the clinical trial proposal screen 61, the row of “number of clinical trial proposals” displays the number of clinical trial proposals for each patient ID. The number of clinical trial proposals is the number of clinical trials in which the patient is a candidate of the subject. When the medical institution selects, for example, “1 case”, a field of “clinical trial proposal details” is displayed in the middle part of the clinical trial proposal screen 61.

The field of “clinical trial proposal details” displays information about the clinical trial in which the patient is a candidate of the subject. If “yes” is displayed in the row of “additional input request” in the corresponding field, it means that there is an item in the patient information 33 of the patient that does not have a value. When the medical institution selects “yes”, a field of “additional input” will be displayed in the lower part of the clinical trial proposal screen 61.

The field of “additional input” displays an additional input request message that “to determine eligibility for clinical trial, please input whether chemotherapy has been administered”. Additionally, the field of “additional input” displays the patient information 33 and the conformance score of the patient. The number such as “[5]” assigned to each item indicates the weight. The row of the item “chemotherapy” is blank due to a missing value. Note that in the above example, the sum of the weights was “1”, but in this case the sum of the weights is “18”.

For example, it is assumed that the medical institution realized that an input has been missed in the past and inputs “no” in this blank. Then, “accept” will be displayed in the row of “accept/reject” in the field of “clinical trial proposal details” in the middle part. On the other hand, for example, there may be cases where a medical institution leaves the blank for maintaining confidentiality for patients. In this case, the medical institution inputs a reason in a row of “reason” in the field of “clinical trial proposal details” in the middle part. Then, “reject” will be displayed in the row of “accept/reject”. Note that after inputting “yes” or “no” in the blank for “chemotherapy” or after inputting a reason in the row of reason, the medical institution presses the “answer” button in the middle part.

As is clear from the above, the proposal unit 23 displays the item for which the value does not exist and prompts input of a value for the item. The extraction unit 22 then compares the values for other items (other than the “whether chemotherapy has been administered”) existing in the patient information with the values for other items in the clinical trial condition, and compares the newly input value for the item in the patient information (for example, “no” for the item “whether chemotherapy has been administered”) with the value in the clinical trial condition corresponding to the newly input value (“no” or “yes” for the same item). In this way, at least some of the additionally input values for the items for which values were missing will be reflected in the conformance score.

Returning to FIG. 6, the flowchart will be described, although a part of description thereof overlaps the description of FIG. 12.

In step S1031, the proposal unit 23 displays the clinical trial proposal screen 61 (FIG. 12) on the medical institution terminal device 3.

In step S1032, the proposal unit 23 determines whether an additional input request is made. Specifically, if “yes” is selected in the field of “additional input request” on the clinical trial proposal screen 61 (step S1032 “YES”), the proposal unit 23 proceeds to step S1033, otherwise (step S1032 “NO”) proceeds to step S1035.

In step S1033, the proposal unit 23 displays the patient information 33 in the field of “additional input” on the clinical trial proposal screen 61.

In step S1034, the proposal unit 23 receives and registers the additional input.

In step S1035, the proposal unit 23 receives the pressing on the “answer” button. Thereafter, the extraction unit 22 reflects the content of the additional input and recalculates the conformance score.

In step S1036, the proposal unit 23 displays to the pharmaceutical company via the output device 13 a message that the missing value has been added. In this case, the proposal unit 23 may display on the output device 13 a screen with contents substantially identical to the clinical trial proposal screen 61 (FIG. 12), or may display on the output device 13 the latest patient retrieval result screen 51 that reflects the content of the additional input. The additional input processing procedure then ends.

### Clinical Trial Retrieval Processing Procedure

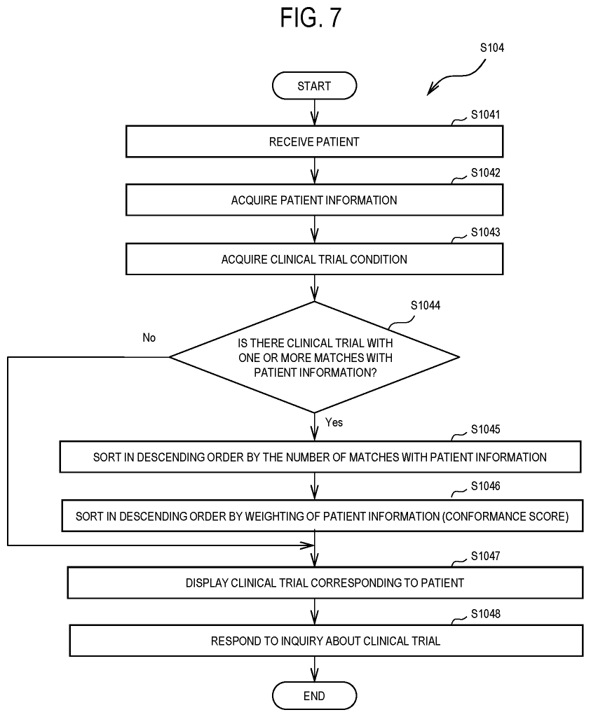

FIG. 7 is a flowchart of a clinical trial retrieval processing procedure. The clinical trial retrieval processing procedure is a detailed description of step S104 in FIG. 3.

In step S1041, the reception unit 21 receives input of a patient as a reference from the medical institution via the medical institution terminal device 3. When calculating the conformance score of all clinical trials for a single patient as a reference, the patient ID of that patient is referred to as a “key patient ID”. A plurality of key patient IDs may be input. For convenience of description, it is assumed here that 10 key patient IDs are input.

In step S1042, the extraction unit 22 acquires the patient information 33 corresponding to the key patient ID from the auxiliary memory device 15.

In step S1043, the extraction unit 22 calculates the conformance score for the combination of the key patient ID and the clinical trial ID by comparing each of the 10 pieces of patient information 33 acquired in step S1042 with the clinical trial conditions 32 of all clinical trials. The number of calculated conformance scores is equal to 10 times the number of clinical trials registered as the clinical trial condition 32. Incidentally, if there is no item in the clinical trial condition 32 that matches the value for the item in the patient information 33, the conformance score will be “0”. The extraction unit 22 stores, for each patient ID, the same number of combinations of the clinical trial ID and the conformance score as the number of clinical trials.

In step S1044, the extraction unit 22 determines whether there is a clinical trial with one or more matches with the patient information. “One or more matches with the patient information” means that there is one or more items with matching values (conformance score is not “0”). If there is a clinical trial with one or more matches with the patient information (step S1044 “YES”), the extraction unit 22 proceeds to step S1045, otherwise (step S1044 “NO”) proceeds to step S1047.

In step S1045, the extraction unit 22 sorts the patient IDs in a descending order of the number of clinical trials with matching values for one or more items in the patient information. That is, the extraction unit 22 arranges the patient IDs in a descending order of the number of candidates of the clinical trial. For example, if there are 40 clinical trials for the patient ID “101”, 50 clinical trials for the patient ID “102”, and 60 clinical trials for the patient ID “103”, the extraction unit 22 will arrange the patient IDs in the order of “103, 102, 101”. The extraction unit 22 may further sort the 40 clinical trials of the patient ID “101” in the order of the number of items in the clinical trial condition that match the items in the patient information. For example, the group of clinical trials with matching values for the four items of “carcinoma”, “gender”, “age”, and “treatment start date” will be ranked in front of the group of clinical trials with matching values for the three items of “carcinoma”, “gender”, and “age”.

In step S1046, the extraction unit 22 sorts the clinical trial IDs in a descending order of the conformance scores. That is, the extraction unit 22 arranges the clinical trial IDs in a descending order of the conformance scores. In the above example, the extraction unit sorts the 60 clinical trial IDs for the patient ID “103” in a decreasing order of the conformance scores, sorts the 50 clinical trial IDs for the patient ID “102” in a decreasing order of the conformance scores, and sorts the 40 clinical trial IDs for the patient ID “101” in a decreasing order of the conformance scores.

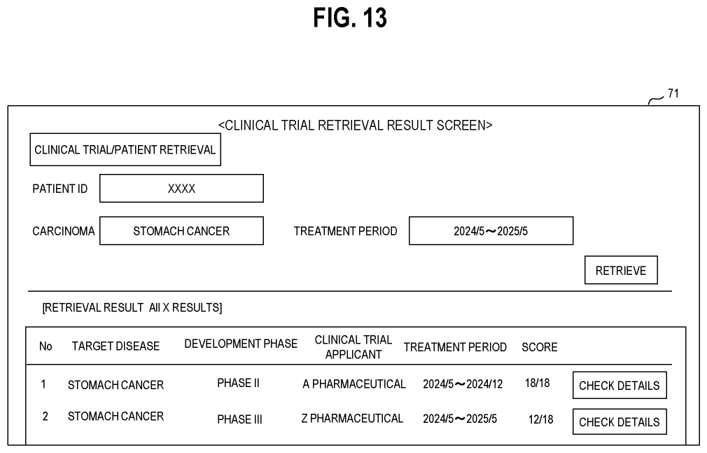

In step S1047, the proposal unit 23 displays candidates of the clinical t corresponding to the patient. Specifically, the proposal unit 23 displays a clinical trial retrieval result screen 71 (FIG. 13) on the medical institution terminal device 3 or the patient terminal device 2. Although description is being made with reference to FIG. 7, the description will be made below with reference to FIG. 13.

FIG. 13 is an example of the clinical trial retrieval result screen 71. The upper part of the clinical trial retrieval result screen 71 displays the key patient ID and information associated therewith. The lower part of the clinical trial retrieval result screen 71 displays a retrieval result. Referring to FIG. 13, for example, the following can be known.
At least two clinical trials were candidates for the patient with the patient ID “XXXX”. Note that in actual, more candidates are displayed.
Of the two candidates, the one with the highest conformance score (18/18) is the clinical trial whose clinical trial applicant (pharmaceutical company) is “A Pharmaceutical”.
The conformance score of the clinical trial whose clinical trial applicant is “Z Pharmaceutical” is the second highest (12/18). Although it is not clear from FIG. 13, the difference in the conformance score with the clinical trial of “A Pharmaceutical” may be occurred due to a mismatch in the values for certain items (age and the like) in the patient information 33.

It is now assumed that a medical institution or a patient presses a “check details” button corresponding to the clinical trial of the “A Pharmaceutical”. The description will be made returning to FIG. 7.

In step S1048, the proposal unit 23 responds to the inquiry about the clinical trial. Specifically, the proposal unit 23 displays questions posed by the medical institution regarding the content of the clinical trial via the output device 13 and receives answers input by the pharmaceutical company via the input device 13. The clinical trial retrieval processing procedure then ends.

### Conformance Score Recalculation Processing Procedure

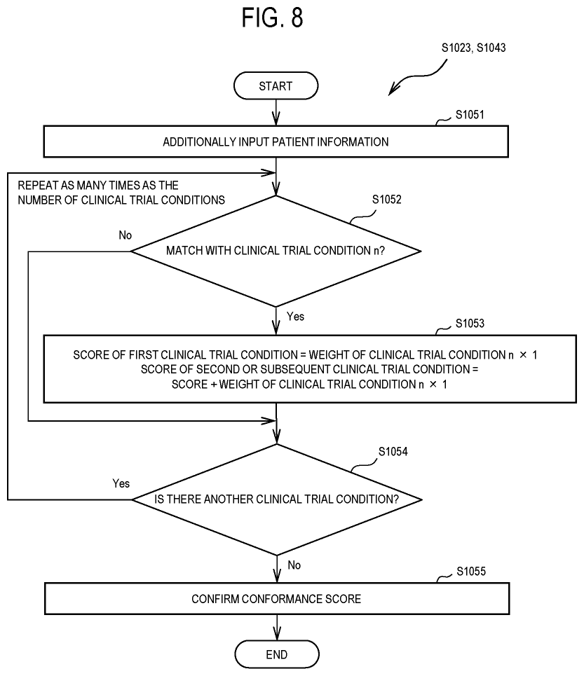

FIG. 8 is a flowchart of a conformance score recalculation processing procedure. The conformance score recalculation processing procedure is processing of recalculating the conformance scores in step S1023 of FIG. 5 and step S1043 of FIG. 7 when a new value is input for an item in the patient information 33 for which the value does not exist.

In step S1051, the reception unit 21 receives the additional input of the patient information 33 from the medical institution via the medical institution terminal device 3. The value to be additionally input here is, for example, “no” in the row of “chemotherapy” in the lower part of FIG. 12. Then, the reception unit 21 updates the patient information 33 based on the additionally input value.

In step S1052, the extraction unit 22 determines whether the value for the nth item (n=1, 2, 3, . . . ) in the patient information 33 matches the value of the nth item in the clinical trial condition 32. If the two values match (step S1052 “YES”), the extraction unit 22 proceeds to step S1053, otherwise (step S1052 “NO”) proceeds to step S1054.

In step S1053, the extraction unit 22 adds up the conformance scores. Specifically, the extraction unit 22 performs the following processing.
When n=1, the extraction unit 22 multiplies the weight of the nth item in the clinical trial condition by the matching degree “1” and sets the result as the conformance score.
When n=2, 3, . . . , the extraction unit 22 multiplies the weight of the nth item in the clinical trial condition by the matching degree “1” and adds the result to the previous conformance score to obtain a new conformance score.

Note that in this example, when the values of the item match, the matching degree “Pn” is “1”. The weight of the nth item in the clinical trial condition corresponds to the aforementioned “Wn”.

In step S1054, the extraction unit 22 determines whether there is a clinical trial condition 32 related to another clinical trial. If there is the clinical trial condition 32 related to another clinical trial (step S1054 “YES”), the extraction unit 22 increments n by “+1” and returns to step S1052, otherwise (step S1054 “NO”) proceeds to step S1055. As a result, the processing of step S1052 and step S1053 is repeated as many times as the number of items n (inner loop not displayed) and as many times as the number of clinical trial conditions 32 (outer loop).

In step S1055, the extraction unit 22 confirms the conformance score. Specifically, the extraction unit 22 determines the latest conformance score as the final conformance score. The conformance score recalculation processing procedure then ends. FIG. 8 shows the processing procedure when the patient information 33 about a specific patient is updated, but the processing procedure before the update is essentially the same.

### Additional Input of Missing Value and Processing Associated Therewith

For example, it is now assumed that there are 10 items in the clinical trial condition and 10 items in the patient information. Naturally, values are set for all items in the clinical trial condition. As mentioned above, the items in the patient information are the same as the items in the clinical trial condition. However, it is assumed that the values for the 9th and the 10th items in the patient information are missing.

In this case, the extraction unit 22 first compares the values of the 1st to the 8th items. For the 9th and the 10th items, the values in the patient information are missing, and therefore the extraction unit 22 cannot make a comparison. Therefore, the extraction unit 22 unavoidably uses ΣWn·Pn (n=1, 2, . . . , 8) as the conformance score.

Next, it is assumed that the proposal unit 23 prompts input of values for the 9th and the 10th items in the patient information. It is assumed that in response to this, the medical institution additionally inputs a value for the 9th item, but does not additionally input a value for the 10th item. In this case, the extraction unit 22 compares the values for the 9th item. That is, the extraction unit 22 calculates W₉·P₉ and adds the result to ΣWn·Pn (n=1, 2, . . . , 8) to obtain the conformance score.

If the value for the 10th item is additionally input at the same time as the value for the 9th item, the extraction unit 22 compares the values for the 9th and the 10th items. That is, the extraction unit 22 calculates W₉·P₉+W₁₀·P₁₀ and adds the result to ΣWn·Pn (n=1, 2, . . . , 8) to obtain the conformance score.

### PS Estimation Processing Procedure

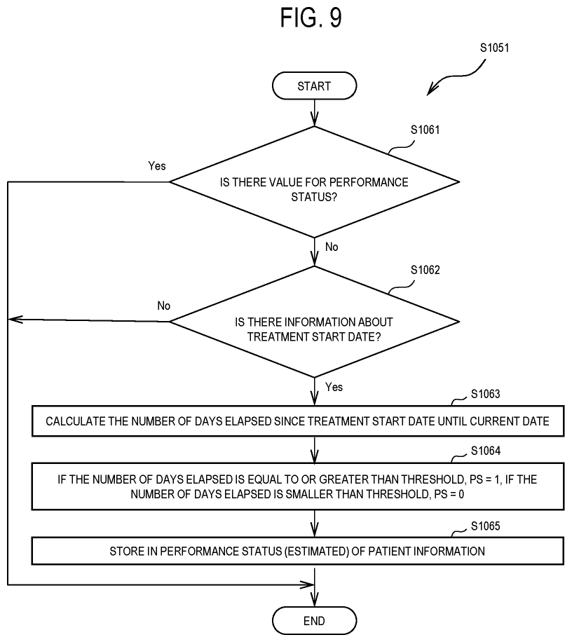

FIG. 9 is a flowchart of a PS estimation processing procedure. It is now assumed that the value for PS does not exist among the items in the patient information 33 about a specific patient, and therefore additional input of this value is requested. In this case, it would be ideal if the medical institution in charge of the patient could additionally input the value. However, the extraction unit 22 can automatically calculate the value for PS based on the treatment start date without waiting for the additional input.

In step S1061, the extraction unit 22 determines whether the value for the item “PS” exists. If the value for PS does not exist (step S1061 “NO”), the extraction unit 22 proceeds to step S1062, and if the value exists (step S1061 “YES”), the extraction unit 22 ends the PS estimation processing procedure.

In step S1062, the extraction unit 22 determines whether the value for the item “treatment start date” exists. If the value for the treatment start date exists (step S1062 “YES”), the extraction unit 22 proceeds to step S1063, and if the value does not exist (step S1062 “NO”), the extraction unit 22 ends the PS estimation processing procedure.

In step S1063, the extraction unit 22 calculates the number of days elapsed since the treatment start date until the current date.

In step S1064, if the number of days elapsed is equal to or greater than a predetermined threshold, the extraction unit 22 determines the value for “PS” to be “1”, and if the number of days elapsed is smaller than the threshold, determines the value for “PS” to be “0”. The extraction unit 22 may determine the value for PS from among a plurality of candidates such as “0, 1, 2, 3, . . . ” by applying the number of days elapsed to a plurality of thresholds.

In step S1065, the extraction unit 22 stores the value determined in step S1064 as the value for the item “PS (estimated)” in the patient information 33. Note that the weight of the item “PS (estimated)” is smaller than the weight of the item “PS”. This is because the values provided by medical institutions that directly examine patients should be paid more attention. The PS estimation processing procedure then ends.

### Ingenuity in Weighting

(1) The extraction unit 22 may receive the importance of each item in the clinical trial condition from the pharmaceutical company and automatically set the weights so that the more important the item, the greater the weight.

(2) If the value for the item “whether anti-cancer drug treatment of XX is being administered” is missing, the extraction unit 22 may refer to the number of days elapsed from a definitive diagnosis until now and adjust the weight of the item for the patient whose number of days elapsed is short. This is because people whose number of days elapsed fewer since the definitive diagnosis are more likely to have little history of medication.

(3) If the value for the item “whether clinical trial in remote location is desired” is missing in the patient information, the extraction unit 22 may adjust the weight of the corresponding item based on an address of the patient and a location of the clinical trial. This is because an economic burden on the patient can be reduced.

It should be avoided that a missing value for a certain item would drastically reduce chances of a patient being a subject. The auxiliary memory device 15 stores information that is associated with an item A whose value is missing and that stores information on an adjustment method for the weight when the value is missing (such as reducing the weight of the item A). The extraction unit 22 refers to the information as appropriate and adjusts the weight.

### Ingenuity in Matching Degree

The matching degree may be a binary value of “1” or “0”, or may be a real number that changes continuously between 0 and 1. For example, when a value for an item is a numerical range or a character string, the extraction unit 22 can calculate the matching degree of the real values based on an overlapping degree between the numerical ranges, an edit distance of characters, or the like.

### Expert Meeting

The proposal unit 23 may display the clinical trial retrieval result screen 71 (FIG. 13) via the medical institution terminal device 3 to the medical institution that conducts a treatment on the candidate of the subject, who is a review target in an expert meeting. In the future, with a consent of the medical institution, the proposal unit 23 may display the clinical trial retrieval result screen 71 (FIG. 13) via the patient terminal device 2 to the candidate of the subject who is the review target in the expert meeting. The expert meeting refers to a meeting for performing medical interpretation based on a result of a cancer gene panel testing and studying the optimal treatment policy, and is also referred to as an “expert panel”. Participants in the expert meeting include cancer chemotherapy specialists, genetic medicine specialists, genetic counseling technicians, pathology specialists, and specialists in molecular genetics and cancer genome medical treatment. Candidates of the subject may be review targets in the expert meeting.

### Effects of Present Embodiment

Effects of the clinical trial proposal device of the present embodiment are as follows.

(1) The clinical trial proposal device can extract the candidate of the subject even when not all values for the items in the patient information are available.

(2) The clinical trial proposal device can supplement missing values.

(3) The clinical trial proposal device can accurately and quantitatively extract the candidate of the subject by utilizing the weights and calculating the conformance score.

(4) The clinical trial proposal device can reconsider the weights.

(5) The clinical trial proposal device can make the expert panel more meaningful.

(6) The clinical trial proposal device can rearrange the candidates of the subject by the number of matches between the items in the patient information and the items in the clinical trial condition.

The invention is not limited to the above-described embodiment, and includes various modifications. For example, the embodiment described above has been described in detail to facilitate understanding of the invention, and the invention is not necessarily limited to those including all the configurations described above. A part of a configuration according to a certain embodiment can be replaced with a configuration according to another embodiment, and a configuration according to another embodiment can be added to a configuration according to a certain embodiment. In addition, another configuration can be added to a part of a configuration of each embodiment, and the part of the configuration of each embodiment can be deleted or replaced with another configuration.

What is claimed is:

1. A clinical trial proposal device comprising:
a reception unit configured to receive a clinical trial condition including a plurality of items required of a subject of a clinical trial, and patient information including the plurality of items about a plurality of patients who may be the subject;
an extraction unit configured to extract, if a value for some item included in the patient information does not exist, a candidate of the subject from the patient information by comparing values for other items existing in the patient information with values for other items in the clinical trial condition, and display the extracted candidate of the subject on a screen; and
a proposal unit configured to propose the clinical trial for the extracted candidate.

2. The clinical trial proposal device according to claim 1, wherein
the proposal unit displays the item for which the value does not exist and prompts input of a value for the item, and
the extraction unit compares the values for other items existing in the patient information with the values for other items in the clinical trial condition, and also compares the newly input value for the item in the patient information with a value in the clinical trial condition that corresponds to the newly input value.

3. The clinical trial proposal device according to claim 1, wherein
the reception unit receives a weight of each of the items, and
the extraction unit calculates a conformance score indicating a degree to which the patient information conforms to the clinical trial condition, as a weighted sum of matching degrees of the values for the items, and extracts a patient with a high conformance score as the candidate of the subject.

4. The clinical trial proposal device according to claim 3, wherein
the reception unit receives the weights after correction, and
the extraction unit recalculates the conformance score based on the weights after correction.

5. The clinical trial proposal device according to claim 1, wherein
the candidate of the subject is a review target in an expert meeting.

6. The clinical trial proposal device according to claim 1, wherein
the extraction unit rearranges the candidate of the subject by the number of the items in the patient information that match the items in the clinical trial condition, and
the proposal unit displays the candidate of the subject in a rearranged order.

7. A clinical trial proposal method comprising:
a reception unit of a clinical trial proposal device receiving a clinical trial condition including a plurality of items required of a subject of a clinical trial, and patient information including the plurality of items about a plurality of patients who may be the subject;
an extraction unit of the clinical trial proposal device extracting, if a value for some item included in the patient information does not exist, a candidate of the subject from the patient information by comparing values for other items existing in the patient information with values for other items in the clinical trial condition, and displaying the extracted candidate of the subject on a screen; and
a proposal unit of the clinical trial proposal device proposing the clinical trial for the extracted candidate.
